(19) United States
(12) Patent Application Publication
(10) Pub. No.: US 2026/0101987 A1
(43) Pub. Date: Apr. 16, 2026

(54) Multifunctional Cosmetic Case with UV Imaging

(71) Applicants: Shenzhen Kaiyan Medical Equipment Co., Ltd., Shenzhen (CN); SHENZHEN NUON MEDICAL EQUIPMENT CO., LTD., Shenzhen (CN)

(72) Inventors: Alain Dijkstra, Amstelveen (NL); Wen Caiyun, Shenzhen (CN); Zhou Hong, Shenzhen (CN); Zhou Jian, Shenzhen (CN); Li Xiang, Shenzhen (CN); Tang Yuanzhou, Shenzhen (CN); Zhou Zhiliang, Shenzhen (CN)

(73) Assignees: Shenzhen Kaiyan Medical Equipment Co., Ltd., Shenzhen (CN); Shenzhen Nuon Medical Equipment Co., Ltd., Shenzhen (CN)

(21) Appl. No.: 19/346,528

(22) Filed: Sep. 30, 2025

(30) Foreign Application Priority Data
Oct. 12, 2024 (CN) 2024224739476
Jan. 20, 2025 (CN) 2025201345154

(51) Int. Cl. A45D 33/32; A45D 33/00; A45D 44/00; A61L 2/10; A61L 2/24; H04N 23/56; H04N 23/57; H04N 23/667

(52) U.S. Cl. CPC A45D 33/32; A45D 33/008; A45D 44/005; A61L 2/10; A61L 2/24; H04N 23/56; H04N 23/57; H04N 23/667; A45D 2044/007; A45D 2200/205; A61L 2202/11; A61L 2202/121; A61L 2202/122; A61L 2202/14; A61L 2202/16

(57) ABSTRACT

The present invention relates to a multifunctional cosmetic box that integrates ultraviolet (UV) imaging for cosmetic application monitoring with applicator sterilization functionality. The device comprises a storage box for cosmetics, a UV lamp, a UV camera, and a display module configured to capture and display UV-based images of a user's face, enabling precise application and removal of products such as sunscreen or foundation. The cosmetic box further includes a sterilization chamber with a light-transmitting window and UV light source for disinfecting makeup applicators, including powder puffs and brushes, thereby preventing bacterial contamination. Sensors and control circuits regulate activation to ensure safe and efficient operation. By combining real-time application monitoring with applicator sterilization in a compact, portable structure, the invention provides improved cosmetic accuracy, enhanced hygiene, and convenience, addressing limitations of conventional cosmetic boxes and puff sterilization devices.

### TECHNICAL FIELD

The present invention relates to the field of cosmetic devices and personal care systems. More specifically, it pertains to a multifunctional cosmetic box that integrates ultraviolet (UV) imaging for monitoring the application or removal of cosmetic products, ultraviolet-based sterilisation for makeup applicators such as powder puffs, sponges, or brushes, and artificial intelligence (AI)-assisted skin analysis within a single compact unit. The invention further relates to portable cosmetic accessories that combine traditional makeup application features, such as mirrors and applicators, with advanced electronic modules for cosmetic monitoring, skin condition assessment, sunscreen evaluation, and applicator disinfection.

### BACKGROUND

Traditionally, cosmetic products are stored in simple cases or boxes designed solely to hold powders, creams, and applicators. While convenient for portability, such cases lack any functionality beyond basic storage. Users are required to rely on separate mirrors, separate UV inspection devices for sunscreen application assessment, and independent sterilization boxes to disinfect makeup applicators. This fragmentation of tools leads to inconvenience, higher cost, and additional space requirements, particularly for users who travel frequently or demand precise cosmetic application.

Conventional cosmetic cases also fail to provide effective solutions for two persistent challenges in skincare and cosmetics. First, users often struggle to apply sunscreen or other products uniformly, as the naked eye cannot easily detect areas of insufficient coverage. This results in inadequate protection and long-term skin damage. Second, applicators such as powder puffs and sponges are prone to bacterial contamination, leading to hygiene issues and potential skin irritation. Existing sterilization boxes, while available, are standalone devices that are not integrated with cosmetic storage or application tools, thereby complicating the user's routine.

Some advanced cosmetic devices have attempted to integrate mirrors with light therapy or UV imaging modules; however, these devices are often bulky, lack cosmetic storage compartments, or fail to incorporate intelligent features such as automated analysis. Users are therefore left without a single, compact, and multifunctional solution that can meet their combined needs for storage, cosmetic application, skin analysis, and applicator hygiene. This gap in the market creates an opportunity for a product that merges traditional cosmetic case functionality with modern technological innovations.

The present invention addresses these limitations by providing a multifunctional cosmetic case that combines cosmetic storage, UV imaging, applicator sterilization, and AI-powered skin analysis into one compact device. By integrating a UV light source, UV camera, and display, the invention enables real-time monitoring of sunscreen and cosmetic application. The inclusion of a UV sterilization module ensures that applicators remain hygienic during storage. Furthermore, the integration of an AI analysis module provides intelligent feedback, such as coverage evaluation, UV index-based SPF recommendations, and predictive tracking of skin conditions. This holistic approach eliminates the need for multiple standalone products, reduces cost, saves space, and enhances the user experience by delivering a portable all-in-one skincare and cosmetic solution.

### OBJECTS OF THE INVENTION

Some of the objects of the present invention are as follows:

An object of the present invention is to provide a multifunctional cosmetic case that integrates storage of cosmetics with ultraviolet (UV) imaging, ultraviolet sterilization, and intelligent analysis functionalities in a single compact housing.

Another object of the present invention is to provide a cosmetic case with a UV imaging unit comprising a UV light source, a UV camera, and a display module, enabling accurate monitoring of cosmetic application, cosmetic removal, or sunscreen coverage on the user's skin.

Another object of the present invention is to provide a cosmetic case with an ultraviolet sterilization module configured to disinfect cosmetic applicators such as powder puffs, sponges, or brushes when stored within the case, thereby improving hygiene and user safety.

Another object of the present invention is to provide a multifunctional cosmetic case with an artificial intelligence (AI) analysis module configured to process UV images to evaluate skin conditions, cosmetic coverage, or sun protection factor (SPF) effectiveness, and to generate predictive models for tracking changes in skin condition over time.

Another object of the present invention is to provide a cosmetic case with a sensor-controlled safety mechanism that automatically deactivates the UV light source and UV camera when the flip cover is closed, thereby preventing unnecessary UV exposure and conserving battery power.

Another object of the present invention is to provide a multifunctional cosmetic case with both a display surface and a cosmetic mirror on opposite sides of the flip cover, allowing the user to selectively employ either UV imaging and analysis or a conventional mirror-based application.

Another object of the present invention is to provide a cosmetic case with a rotatable flip cover and a rotatable camera area, allowing the user to adjust the position and orientation of the UV camera or alternate between the inner and outer surfaces according to their needs.

Another object of the present invention is to provide a cosmetic case with an integrated feedback interface, including both display-based visual feedback and speaker-based audio prompts, to guide the user in real time during cosmetic application, removal, and skincare monitoring.

Another object of the present invention is to provide a cosmetic analysis system configured with AI-driven colour inversion imaging to enhance the visibility of cosmetic residues and uneven applications, particularly across different skin tones.

Another object of the present invention is to provide a multifunctional cosmetic case capable of measuring ambient UV index exposure, recommending appropriate SPF usage, and reminding the user about sunscreen reapplication through integrated AI and feedback systems.

Another object of the present invention is to provide a multifunctional cosmetic case that is portable, user-friendly, and capable of replacing separate cosmetic mirrors, UV imaging devices, and standalone sterilization boxes, thereby reducing cost, saving space, and enhancing the overall user experience.

### SUMMARY OF THE INVENTION

According to a first aspect of the present invention, a multifunctional cosmetic case is provided. The case comprises: a base box configured to store one or more cosmetic products; a flip cover coupled to the base box; an ultraviolet (UV) light source positioned on the flip cover to direct UV light toward a user's skin; a UV camera configured to capture one or more images of the user's skin irradiated by the UV light; and a display module operatively connected to the UV camera and arranged to display the captured UV image. In this arrangement, the cosmetic case enables monitoring of the application or removal of cosmetic products on the skin.

In one embodiment of the invention, the cosmetic case further comprises an artificial intelligence (AI) analysis module configured to process the images captured by the UV camera to evaluate cosmetic coverage, skin condition, or sun protection factor (SPF) effectiveness. The AI analysis module may cooperate with a feedback interface provided on the display module to deliver visual feedback, audio feedback, or a combination thereof.

In one embodiment of the invention, the cosmetic case further comprises a sensor configured to automatically deactivate the UV light source and the UV camera when the flip cover is closed. In another embodiment, the cosmetic case further comprises a speaker operatively connected to the control circuit to provide audio prompts and feedback, such as application reminders, sterilization completion alerts, or cosmetic guidance.

According to a second aspect of the present invention, a multifunctional cosmetic case is provided comprising: a base box having a storage chamber for a cosmetic applicator; a UV sterilization module comprising one or more UV light sources arranged to emit UV light into the storage chamber through a light-transmitting window; a flip cover having a UV camera and a display module on a first side, the UV camera is configured to capture one or more images of the user's skin irradiated by the UV light; and the display module is operatively connected to the UV camera to display the captured one or more images; and a control circuit configured to activate the UV sterilization module when the applicator is enclosed in the storage chamber. In this configuration, the cosmetic applicator is disinfected during storage.

In one embodiment of the invention, the flip cover further comprises a cosmetic mirror provided on a second side. In another embodiment, the UV sterilization module is configured to emit UV light from both a bottom wall and a circumferential wall of the storage chamber, thereby achieving multi-directional sterilization. In some embodiments, the flip cover includes a rotatable camera area, allowing adjustment of the UV camera orientation relative to the user.

In one embodiment of the invention, the artificial intelligence analysis module is further configured to perform color inversion imaging to enhance the visibility of cosmetic residues or uneven application. In another embodiment, the AI analysis module evaluates UV index exposure, recommends SPF usage, and employs predictive modeling to track changes in a user's skin condition over time.

In one embodiment of the invention, the multifunctional cosmetic case further comprising a speaker electrically connected to the control circuit for providing audio feedback.

In one embodiment of the invention, the flip cover is mounted on one or more shafts, allowing two-axis rotation between the first side and the second side.

In one embodiment of the invention, the flip cover comprises a rotatable area with the first side and the second side, and the flip cover is configured to interchange the position of the first side and the second side through rotational movement.

In one embodiment of the invention, the flip cover further comprising a rotatable camera allowing adjustment of the UV camera orientation relative to the user.

In one embodiment of the invention, the UV camera is detachable from the multifunctional cosmetic case and operatively connectable via a wired or wireless interface.

According to a third aspect of the present invention, a method of cosmetic application and skin analysis using a multifunctional cosmetic case is provided. The method comprises: storing at least one cosmetic product in the cosmetic case; irradiating UV light toward a user's face; capturing one or more images of the face with a UV camera; processing the images with an AI analysis module to determine cosmetic distribution, skin features, or SPF effectiveness; and displaying the processed results on a display module.

In one embodiment of the invention, the method further comprises measuring the ambient UV index and recommending an SPF value to the user. In another embodiment, the method includes performing color inversion imaging to enhance the visibility of cosmetic residues or uneven cosmetic application. In yet another embodiment, predictive modeling is employed to track variations in a user's skin condition over time, thereby providing long-term dermatological monitoring.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “flexible substrate” refers to a base material that supports the placement and interconnection of electrical components, while allowing the overall structure to bend or conform to non-planar surfaces. Examples include polyimide film, thermoplastic elastomers, and silicone-based sheets.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a multifunctional cosmetic case, in accordance with an embodiment of the present invention.

FIG. 2 illustrates an exploded view of the multifunctional cosmetic case, in accordance with an embodiment of the present invention.

FIG. 3 illustrates a front view of a cover frame of the multifunctional cosmetic case, in accordance with an embodiment of the present invention.

FIG. 4 illustrates a front view of the structure of an inner shell of the multifunctional cosmetic case, in accordance with an embodiment of the present invention.

FIG. 5 illustrates a front view of the assembly of the inner shell and a tray of the multifunctional cosmetic case, in accordance with an embodiment of the present invention.

FIG. 6 illustrates a perspective view of an alternate configuration of a multifunctional cosmetic case, in accordance with an embodiment of the present invention.

FIG. 7 illustrates a top perspective exploded view of the multifunctional cosmetic case, in accordance with an embodiment of the present invention.

FIG. 8 illustrates a bottom perspective exploded view of the multifunctional cosmetic case, in accordance with an embodiment of the present invention.

FIG. 9 illustrates a cross-sectional view of the multifunctional cosmetic case, in accordance with an embodiment of the present invention.

FIG. 10 illustrates a cross-sectional view of the multifunctional cosmetic case showing the placement of a hall sensor and magnet, in accordance with an embodiment of the present invention.

FIG. 11 illustrates another top perspective exploded view of the multifunctional cosmetic case, in accordance with an embodiment of the present invention.

FIGS. 12A and 12B illustrate a perspective view of different orientations of a flip cover of the multifunctional cosmetic mirror, in accordance with an embodiment of the present invention.

FIG. 13 illustrates a perspective view of the different orientations of a display area and the flip cover of the multifunctional cosmetic mirror, in accordance with an embodiment of the present invention.

FIGS. 14A and 14B illustrate a perspective view of the different orientations of a camera area of the multifunctional cosmetic mirror, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention will now be described in detail with reference to the accompanying drawings, wherein like reference numerals designate like or corresponding components throughout. The embodiments illustrate a multifunctional cosmetic device that integrates a mirror surface, a phototherapy module, and an ultraviolet (UV) imaging system into a single compact housing. The device is configured with two opposite functional faces, each adapted to provide distinct cosmetic and therapeutic benefits while sharing common structural and control elements.

Embodiments of the present invention disclose a multifunctional cosmetic mirror device configured to combine conventional cosmetic mirror functionality with imaging and phototherapy capabilities for enhanced skin analysis and treatment. The cosmetic mirror device comprises a mirror frame having at least a first side and a second side. A mirror surface is mounted on the first side of the frame and is configured for use as a cosmetic mirror by a user. At least one functional module is mounted on the frame, the module being selected from an imaging module configured to analyze a skin condition of the user and a phototherapy module configured to apply therapeutic light to the skin. In certain embodiments, both the imaging module and the phototherapy module are provided within the same device, and a control unit is arranged to selectively activate one or both modules depending on the operational mode.

In some embodiments, the second side of the mirror frame is provided with both the ultraviolet (UV) imaging module and the phototherapy module. In such configurations, the UV imaging module may be employed to analyze subsurface skin features under UV illumination, while the phototherapy module may deliver therapeutic light such as red, blue, or infrared wavelengths to the skin. The arrangement of both modules on the second side of the frame allows the device to function as a dedicated diagnostic and therapeutic unit when rotated, thereby extending its versatility beyond conventional mirror use and providing the user with comprehensive skincare analysis and treatment capabilities within a single device.

In one embodiment, the phototherapy module is arranged on the same side of the frame as the mirror surface. The phototherapy module may be disposed at the side of the mirror surface, behind the mirror surface, or directly on or adjacent to the mirror surface. The phototherapy module is configured to irradiate UV light, infrared light, blue light, or red light. In a preferred embodiment, the phototherapy module comprises an infrared light-emitting assembly configured to irradiate invisible infrared light toward the user while the mirror surface reflects a visible image. This arrangement enables a user to simultaneously view their reflection in the mirror while receiving phototherapy treatment without visual interference. The infrared light-emitting assembly may include one or more infrared light-emitting diodes (LEDs) operating in the wavelength range of approximately 700 nm to 1100 nm.

A distance sensor may be provided in proximity to the mirror surface to detect the spacing between the user and the mirror. The distance sensor is coupled to the control circuit such that operation of the phototherapy module is enabled or disabled based on whether the detected spacing falls within a predetermined safe range. This ensures safe and reliable delivery of phototherapy by preventing overexposure when the user's face is too close to the mirror surface.

In certain embodiments, the phototherapy module is configured to emit light in the far-infrared spectrum, which is particularly effective for promoting skin rejuvenation and stimulating deeper tissue layers while not interfering with the user's visibility during mirror use. During conventional operation of the mirror, far-infrared light is preferably applied so that the user may simultaneously perform cosmetic application and receive therapeutic benefits.

The device may further be configured to dynamically adjust the spectral range and intensity of emitted radiation based on the spacing between the user and the mirror, as determined by the distance sensor. For example, when the user is positioned closer to the mirror, the control circuit may automatically reduce the emission intensity or switch the emission to near-infrared wavelengths to ensure safe exposure. Conversely, when the user is located at a greater distance, the control circuit may allow higher intensity or switch to far-infrared output to maintain therapeutic efficacy. This adaptive control of wavelength and intensity provides a personalized and safe phototherapy experience while maximizing treatment effectiveness.

In another embodiment, the multifunctional cosmetic device is implemented as a dual-sided frame. The first side of the frame comprises the mirror surface, a visible light imaging unit, and the phototherapy module, while the second side of the frame comprises an ultraviolet (UV) imaging unit. The UV imaging unit may include a UV lamp and a UV-sensitive camera to capture and display UV images of the user's skin, thereby allowing detection of hidden blemishes, pigmentation, or sun damage. An activation mechanism is integrated within the frame and coupled to the control circuit to selectively activate either the visible light imaging unit and the phototherapy module when the first side faces the user, or the UV imaging unit when the second side faces the user. The activation mechanism may be a mechanical limit switch actuated by rotation of the frame, or an orientation sensor that automatically detects which side of the frame is oriented toward the user. The control circuit may further be configured to prevent simultaneous activation of the UV imaging unit and the phototherapy module to ensure operational safety.

A fill-light module may optionally be arranged circumferentially around the mirror surface to provide uniform illumination for cosmetic application or imaging purposes. In certain embodiments, the visible light imaging unit and the phototherapy module may operate simultaneously, enabling both visual reflection of the user and concurrent phototherapy treatment. The device may further be configured with a user interface to control light intensity, duration of treatment, and switching between imaging modes.

In another embodiment of the present invention, a method of operating a multifunctional cosmetic mirror device is provided. The method enables simultaneous cosmetic mirror use and phototherapy treatment under safe operating conditions.

In one embodiment, the method comprises providing a mirror surface on a first side of a frame, the mirror surface being configured to reflect a visible image of a user. An infrared light-emitting assembly is disposed in association with the mirror surface and is activated during operation. When the user positions their face in front of the mirror, the mirror surface reflects the visible image of the user, while at the same time, the phototherapy module provides phototherapy to the user's skin. This arrangement enables the user to view their reflection in real time without visual interference, while receiving therapeutic exposure to infrared wavelengths. The phototherapy module is configured to irradiate red light, blue light, or infrared light. In a preferred embodiment, the phototherapy module is configured to irradiate infrared light, enabling a user to simultaneously use the mirror as well as receive phototherapy.

The method further comprises detecting, by means of a distance sensor, a spacing between the user and the mirror surface. The distance sensor generates a distance signal that is communicated to a control circuit. The control circuit is operatively coupled to the infrared light-emitting assembly and regulates its operation based on the detected spacing. When the user is located within a predetermined safe distance range, the control circuit permits activation of the infrared assembly. If the detected spacing falls below a safe minimum threshold, the control circuit disables the infrared emission to prevent overexposure. In this way, user safety is maintained while still allowing efficient phototherapy.

In certain embodiments, the method includes controlling specific operational parameters of the infrared assembly, such as the irradiation wavelength, intensity, or duration of exposure. The infrared light-emitting assembly may comprise one or more infrared LEDs configured to emit within the wavelength range of approximately 700 nm to 1100 nm, thereby targeting skin layers most suitable for cosmetic and therapeutic treatment.

In another embodiment, the method further comprises rotating the frame such that a second side having an ultraviolet (UV) imaging unit faces the user. In this position, the control circuit automatically disables the infrared assembly and enables the UV imaging unit. The UV imaging unit may capture and display UV images of the user's skin, allowing analysis of subsurface pigmentation, blemishes, or sun damage. Switching between the two operational modes: infrared phototherapy and UV imaging, is thus achieved seamlessly based on the orientation of the frame, thereby simplifying user interaction.

The multifunctional cosmetic device described herein offers several advantages over conventional cosmetic mirrors and standalone imaging or therapy devices. It integrates mirror, imaging, and phototherapy functionalities into a single compact unit, thereby reducing cost and space requirements. By providing simultaneous visible reflection and invisible infrared therapy, the device supports convenient cosmetic application while delivering skin treatment. The inclusion of UV imaging on a second side of the frame extends the functionality to diagnostic use, enabling users to assess underlying skin conditions. Safety features such as distance sensing and exclusive activation control further enhance user confidence and reliability of operation.

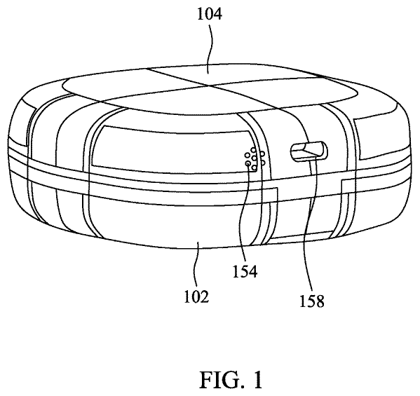

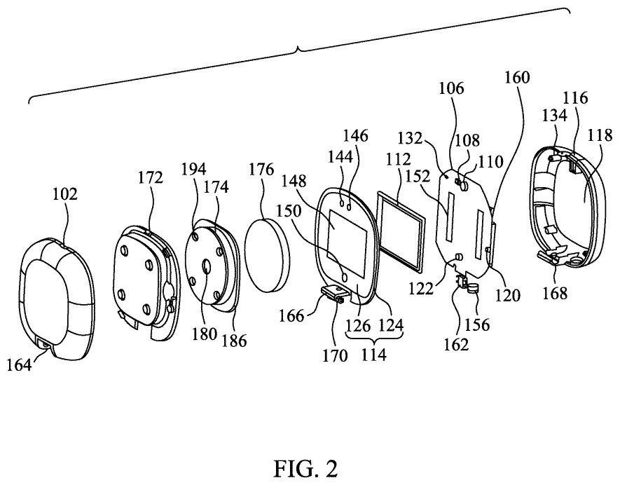

Referring to FIGS. 1 and 2, in an embodiment of the present invention, a multifunctional cosmetic case is provided that is designed to integrate cosmetic storage with ultraviolet (UV) imaging functionality for enhanced cosmetic application. The cosmetic case generally comprises a base box 102, a flip cover 104, a circuit board 106, one or more UV lamp beads 108, a UV camera 110, and a display module 112. Each of these elements is arranged in cooperation with one another to form a compact, portable, and multifunctional device.

The base box 102 defines a main storage compartment for accommodating various cosmetic products. In practical applications, the base box 102 may be dimensioned to store a plurality of cosmetic items such as sunscreen, foundation, eye cream, moisturizer, or other topical skincare products. The base box 102 provides the user with a convenient storage solution, enabling cosmetics to be carried together with the imaging and monitoring system in an integrated case. The inner surface of the base box 102 may include supporting trays, partitions, or recesses to secure individual cosmetic containers, although such details are not limited to the scope of the present invention.

The cosmetic case further comprises a flip cover 104 that is coupled to the base box 102 in an openable and closable manner. The flip cover 104 serves both as a protective enclosure for the contents of the base box 102 during non-use and as a functional housing for electronic and optical components when in operation. The flip cover 104 comprises a translucent cover plate 114 and a cover box 116. The translucent cover plate 114 is disposed on a side of the cover box 116 adjacent to the base box 102. Together, the translucent cover plate 114 and the cover box 116 define a receiving cavity 118. The receiving cavity 118 provides an enclosed space within the flip cover 104 for accommodating electronic and optical components, thereby ensuring both protection and compact integration of the functional modules.

Within the receiving cavity 118, a circuit board 106 is disposed. The circuit board 106 acts as the central electronic platform, supporting and electrically interconnecting the UV lamp beads 108, the UV camera 110, and the display module 112. In one exemplary embodiment, the circuit board 106 may further incorporate control circuits, power management modules, and driver circuits necessary for regulating the operation of the UV light source, image acquisition, and display output. The arrangement of the circuit board 106 within the receiving cavity 118 allows the components to be securely mounted while maintaining a compact structure.

The UV lamp beads 108 are mounted on the circuit board 106 such that they face the translucent cover plate 114. Each UV lamp bead 108 functions as a discrete ultraviolet light source capable of emitting UV radiation through the translucent cover plate 114. In operation, the UV light passes through the translucent cover plate 114 and is directed toward the user's skin, particularly the face. The translucent cover plate 114, being fabricated from a material with high ultraviolet transmittance, ensures minimal attenuation of the emitted UV light. The UV lamp beads 108 are preferably arranged in a manner that achieves uniform irradiation across the desired imaging area, thereby ensuring that the reflected UV light from the skin surface is sufficient for accurate imaging.

Adjacent to the UV lamp beads 108, the UV camera 110 is likewise mounted on the circuit board 106 and oriented toward the translucent cover plate 114. The UV camera 110 is configured to capture reflected UV light from the surface of the user's face after irradiation. By detecting the reflected UV signal, the UV camera 110 generates a facial image in the ultraviolet spectrum. Such imaging reveals cosmetic application characteristics that are not discernible under visible light, such as uneven distribution of sunscreen, incomplete makeup removal, or residual impurities on the skin surface. The UV camera 110 is electrically coupled to the circuit board 106, through which image data is transmitted for further processing or display.

The display module 112 is disposed between the translucent cover plate 114 and the circuit board 106. The display module 112 is electrically connected to the circuit board 106 and is configured to present the images captured by the UV camera 110. In some embodiments, the display module 112 may be realized as a liquid crystal display (LCD), an organic light-emitting diode (OLED) screen, or any equivalent high-resolution display panel. Placement of the display module 112 in direct proximity to the translucent cover plate 114 allows the user to conveniently observe the captured image while using the cosmetic case. This enables real-time visual feedback, thereby assisting the user in applying or removing cosmetic products more precisely.

In practical operation, the user may first open the flip cover 104 to expose the base box 102 and access the stored cosmetic products. During makeup application, the flip cover 104 remains open, and the UV lamp beads 108 are activated to emit UV light toward the user's face through the translucent cover plate 114. The reflected UV light is captured by the UV camera 110, and the corresponding image is displayed on the display module 112. The user can thus observe, in real time, the distribution of applied cosmetic products and adjust application as necessary to achieve uniform coverage. When the cosmetic case is not in use, the flip cover 104 may be closed, thereby enclosing the electronic components within the receiving cavity 118 and protecting the stored cosmetics within the base box 102.

In an embodiment, the multifunctional, portable cosmetic case is provided, wherein the structural integration of the base box 102, flip cover 104, translucent cover plate 114, receiving cavity 118, circuit board 106, UV lamp beads 108, UV camera 110, and display module 112 provides both storage functionality and UV-based cosmetic application monitoring.

The display module 112 is positioned between the translucent cover plate 114 and the circuit board 106. In an embodiment, the display module 112 may be provided as a display screen, such as a liquid crystal display (LCD) or an organic light-emitting diode (OLED) panel, although the invention is not limited thereto. The primary function of the display module 112 is to present, in real time, the facial images acquired by the UV camera 110. By situating the display module 112 in proximity to the translucent cover plate 114, the arrangement ensures a compact configuration while allowing the user to directly observe imaging results without requiring an external device.

When the user intends to perform cosmetic application or removal, the flip cover 104 of the cosmetic case is opened to expose the base box 102 and activate the electronic modules. Upon activation, the UV lamp beads 108 emit ultraviolet light, which propagates through the translucent cover plate 114 and irradiates the user's face. The translucent cover plate 114 is fabricated from a material of high ultraviolet transmittance, thereby ensuring that the emitted UV light passes through with minimal attenuation and retains sufficient intensity for imaging purposes.

As the ultraviolet light impinges upon the surface of the skin, the degree of reflection varies across different facial regions. Such variation is directly influenced by the presence, absence, or thickness of applied cosmetic products such as sunscreen, foundation, or other topical substances. Areas with thicker or more evenly distributed cosmetic layers reflect UV light differently compared to areas with little or no cosmetic coverage. These reflection differences create a distinctive pattern of contrast across the facial surface under ultraviolet illumination.

The UV camera 110, oriented toward the translucent cover plate 114, is configured to capture these differential reflections with high sensitivity. In doing so, the UV camera 110 generates an image that represents the actual distribution of cosmetics across the face. Regions where sunscreen has been adequately applied appear distinctly from regions with insufficient or absent coverage. The generated image is transmitted, via the circuit board 106, to the display module 112 for presentation to the user.

The user, by observing the displayed image on the display module 112, can readily determine which facial areas have been evenly coated with sunscreen or foundation and which areas require additional application or adjustment. The imaging feedback thus provides an objective, visual mechanism for ensuring even cosmetic coverage. Unlike traditional cosmetic application methods, which rely solely on subjective judgment in visible light conditions, the present embodiment allows users to make data-driven corrections based on ultraviolet reflection patterns.

It is readily understood that during cosmetic application, inconsistent reflections between applied and unapplied regions result in correspondingly inconsistent images captured by the UV camera 110. For example, areas lacking sunscreen may appear darker or less reflective, while areas with sufficient coverage exhibit a distinct reflective signature. By evaluating these differences in the displayed image, the user may ascertain both the coverage area and the thickness of cosmetic products. This enables a more accurate determination of whether cosmetic products, such as sunscreen, have been applied adequately to the forehead, cheeks, nose, or other regions of the face.

Through this feedback mechanism, the multifunctional cosmetic case provides a significant technical advantage. Users are empowered to exercise precise control over the application and removal of cosmetics, thereby ensuring uniformity and completeness of coverage. This not only improves the aesthetic outcome of cosmetic use but also enhances the functional performance of products such as sunscreen, which require even distribution to provide effective ultraviolet protection. Furthermore, the same mechanism assists in verifying thorough removal of makeup, thereby reducing the risk of residual buildup, clogged pores, or skin irritation.

In an embodiment, a cosmetic case integrates the UV lamp beads 108, UV camera 110, and display module 112 in a compact arrangement that delivers clear and intuitive visualization of cosmetic distribution on the skin. The multifunctional cosmetic case, therefore, assists the user in both application and removal processes, improving cosmetic effectiveness, safety, and user confidence. Additionally, because all components are structurally and functionally integrated into a single portable cosmetic case, a compact and lightweight form factor is maintained, thereby ensuring portability without sacrificing functionality.

Referring to FIG. 2, in an embodiment, the multifunctional cosmetic case further comprises a sensor 120 electrically connected to the circuit board 106. The sensor 120 functions as an automatic control element configured to manage the operational state of the cosmetic case in response to the position of the flip cover 104 relative to the base box 102. Specifically, the sensor 120 is adapted to shut down the active components of the cosmetic case when the flip cover 104 is moved into a closed position over the base box 102.

In an embodiment, the sensor 120 may be embodied as a proximity sensor, an optical sensor, a hall sensor, or an equivalent detection device capable of identifying positional changes between the flip cover 104 and the base box 102. For example, the sensor 120 may detect closure by monitoring the passage or blocking of light as the flip cover 104 approaches the base box 102. When the sensor 120 confirms that the flip cover 104 is fully closed, it transmits a control signal to the circuit board 106, instructing the system to deactivate operational components such as the UV lamp beads 108, the UV camera 110, and the display module 112. In this manner, the multifunctional cosmetic case achieves automatic shutdown without requiring manual user intervention.

The adoption of the sensor 120 provides multiple technical advantages. First, the automatic shutdown function reduces unnecessary power consumption, thereby extending the operational life of an integrated rechargeable battery. Second, the automation simplifies user interaction, as the user only needs to close the flip cover 104 after each use to ensure that the system is fully powered off. Finally, automatic deactivation improves safety by preventing inadvertent operation of ultraviolet components when the cosmetic case is closed, thus reducing the risk of unnecessary UV exposure.

In addition to the automatic shutdown mechanism, the multifunctional cosmetic case also incorporates a touch switch 122. The touch switch 122 is electrically connected to the circuit board 106 and is arranged on the side of the circuit board 106 facing the translucent cover plate 114. The touch switch 122 serves as a power-on control mechanism for the cosmetic case.

In an embodiment, the touch switch 122 may be implemented as a non-contact sensor switch, such as an infrared touch switch. When the user intends to activate the cosmetic case, they may simply touch or place their finger on the area of the translucent cover plate 114 corresponding to the position of the infrared touch switch 122. The presence of the user's finger or hand interrupts the passage of infrared light, resulting in a detectable change in light intensity. The circuit board 106, upon receiving this change in signal from the touch switch 122, interprets it as a touch action and issues a command to power on the electronic components. As a result, the UV lamp beads 108, UV camera 110, and display module 112 are simultaneously activated, rendering the cosmetic case operational.

This arrangement eliminates the need for mechanical buttons, thereby simplifying operation and reducing mechanical wear. Moreover, the touch switch 122 improves the waterproofing and sealing properties of the cosmetic case, since no external moving parts penetrate the housing. Accordingly, the integration of the touch switch 122 provides an intuitive, user-friendly, and reliable method for powering on the cosmetic case.

Referring further to FIGS. 2 and 3, the translucent cover plate 114 is itself constructed as a composite assembly comprising a cover frame 124 and a light-transmitting plate 126. The cover frame 124 is mechanically connected to the cover box 116 and includes a receiving groove 128. The receiving groove 128 is formed to accommodate multiple electronic components, and its lower portion is provided with a plurality of receiving holes specifically dimensioned for housing the UV lamp beads 108, UV camera 110, display module 112, and touch switch 122, respectively.

To achieve a secure and stable assembly, the four corners of the receiving groove 128 are each provided with a first connection hole 130. Correspondingly, the circuit board 106 is provided with second connection holes 132, each aligned with the first connection holes 130. In addition, the cover box 116 is provided with a plurality of connection posts 134 aligned with the second connection holes 132. By inserting fasteners, such as screws, through the aligned holes and into the connection posts 134, a fixed assembly of the cover frame 124, the circuit board 106, and the cover box 116 is achieved. This fastening arrangement ensures precise positioning, mechanical stability, and long-term durability of the integrated components.

The receiving groove 128 further comprises dedicated openings tailored for the installation of individual functional elements. Specifically, an upper portion of the receiving groove 128 is provided with a first receiving hole 136 designed for the UV lamp beads 108. Adjacent to this, a second receiving hole 138 is provided for the UV camera 110. A third receiving hole 140, located centrally within the receiving groove 128, is dimensioned for housing the display module 112. Finally, a fourth receiving hole 142, situated in the lower portion of the receiving groove 128, is designed for the touch switch 122. The size, shape, and positioning of each receiving hole are precisely matched to the corresponding component to ensure accurate alignment, proper optical functioning, and secure fixation.

Once the electronic components are positioned within the receiving groove 128, the light-transmitting plate 126 is mounted over the groove. The light-transmitting plate 126 covers the entire housing region, thereby shielding and protecting the electronic components contained within. The light-transmitting plate 126 is fabricated from a transparent or semi-transparent material that allows passage of ultraviolet light and visible light, thereby ensuring that the UV lamp beads 108 can irradiate outward, the UV camera 110 can capture reflected signals, and the display module 112 can be viewed clearly by the user. At the same time, light-transmitting plate 126 provides a robust protective barrier, safeguarding sensitive electronic components from dust, moisture, and mechanical impact.

Through this integrated construction, the cosmetic case achieves a compact, secure, and functional design, wherein all critical electronic components are precisely positioned and mechanically reinforced. The combined use of the cover frame 124, receiving groove 128, the first connection holes 130, the second connection holes 132, the connection posts 134, and the protective light-transmitting plate 126 not only simplifies assembly but also enhances structural rigidity, thereby contributing to long-term stability and durability of the multifunctional cosmetic case.

During operation of the multifunctional cosmetic case, the UV lamp beads 108 emit ultraviolet light, which passes outward through the light-transmitting plate 126 and irradiates the face of the user. The UV camera 110, arranged in proximity to the UV lamp beads 108, captures the ultraviolet light reflected from the user's skin through the light-transmitting plate 126, and generates a corresponding facial image based on the reflection pattern. The reflected signals exhibit contrast depending on the thickness and uniformity of cosmetic application, thereby enabling the UV camera 110 to distinguish regions of adequate coverage from those lacking sufficient product. The generated image is then transmitted to and displayed on the display module 112, allowing the user to intuitively determine the status of cosmetic application or removal.

The touch switch 122, disposed in alignment with the translucent cover plate 114, senses user interaction through the light-transmitting plate 126. A touch action is detected when the user makes contact with or obstructs the touch area 150, thereby altering the detected light intensity or capacitance. In response, the touch switch 122 communicates with the circuit board 106 to control the power-on state of the cosmetic case. This design eliminates the need for mechanical switches, thereby enhancing durability, waterproofing, and ease of operation.

In the present embodiment, the multifunctional cosmetic case makes advantageous use of the receiving groove 128, the first receiving holes 136, the second receiving holes 138, the third receiving holes 140, and the fourth receiving holes (136, 138, 140, 142) formed therein. Each electronic component, including the UV lamp beads 108, the UV camera 110, the display module 112, and the touch switch 122, is tightly and orderly arranged within the receiving groove 128 and its associated receiving holes. This configuration achieves precise alignment, secure fixation, and compact integration of all elements, thereby saving internal space and contributing to a lightweight and portable overall structure. The orderly arrangement also facilitates manufacturing consistency, maintenance, and assembly efficiency.

In an embodiment, the light-transmitting plate 126 is further subdivided into functional zones that correspond to individual electronic components. Specifically, the light-transmitting plate 126 is provided with a light-transmitting area 144 corresponding to the UV lamp beads 108, a camera area 146 corresponding to the UV camera 110, a display area 148 corresponding to the display module 112, and a touch area 150 corresponding to the touch switch 122.

In operation, ultraviolet light emitted by the UV lamp beads 108 passes directly through the light-transmitting area 144 with minimal attenuation, ensuring adequate facial illumination. The UV camera 110 captures the reflected ultraviolet light through the camera area 146, which is designed to maximize optical transparency while protecting the sensor. The display module 112 presents the facial image to the user through the display area 148, ensuring high visibility and image clarity. Finally, the user engages the touch switch 122 via the touch area 150, thereby intuitively controlling the activation of the cosmetic case.

The light-transmitting plate 126 integrates distinct functional areas, such as a light-transmitting area 144, a camera area 146, a display area 148, and a touch area 150, which improve user experience by allowing intuitive identification of each control or output region. The user can visually recognize and operate the corresponding areas without requiring external instructions, thereby simplifying interaction and increasing ease of use.

Furthermore, the multifunctional cosmetic case additionally incorporates a cushioning pad 152 disposed between the display module 112 and the circuit board 106. In this embodiment, the cushioning pad 152 is provided on the back surface of the display module 112. The cushioning pad 152 may be a foam strip or other resilient material, and the number of pads may be varied depending on design requirements, such as two pads symmetrically positioned. The cushioning pad 152 serves multiple functions: it stabilizes the display module 112, prevents vibration or displacement during handling, and creates an air gap between the display module 112 and the circuit board 106. The air gap facilitates heat dissipation, thereby preventing overheating and ensuring the long-term safety, reliability, and performance stability of the electronic components.

According to an embodiment of the invention, the cover box 116 is further provided with at least one sound-transmitting hole 154. A speaker 156 is electrically connected to the circuit board 106 and positioned in correspondence with the sound-transmitting hole 154. In one exemplary configuration, the sound-transmitting holes 154 may be arranged in multiples and formed as fine perforations or narrow slits. This arrangement permits efficient transmission of sound from the speaker 156 while maintaining the structural integrity and aesthetic design of the cover box 116.

The speaker 156 is mounted on the circuit board 106 and aligned with the sound-transmitting hole 154 to ensure effective propagation of audio signals to the external environment. In use, the speaker 156 provides intuitive audio prompts to enhance interactivity and usability. For example, when the user activates the cosmetic case by engaging the touch switch 122, the speaker 156 may emit a preset startup sound, indicating successful power-on. When the flip cover 104 is closed and the cosmetic case powers down, the speaker 156 may emit a shutdown sound, confirming deactivation. Furthermore, after the UV camera 110 captures and processes a facial image, the speaker 156 may provide an audio notification indicating that the image capture process is complete and results are available for viewing on the display module 112.

The provision of the sound-transmitting holes 154 in combination with the speaker 156 thus enables the multifunctional cosmetic case to deliver clear and direct audio feedback. This enhances the overall interactive experience, improves ease of operation, and supports accessibility for users who may rely on auditory signals in addition to visual cues. The integration of audio functionality within the cosmetic case represents a further step toward creating a highly interactive, user-friendly, and multifunctional cosmetic device.

In an embodiment, the cover box 116 is further provided with a plug-in port 158 configured for power input. A rechargeable battery 160 is electrically connected to the side of the circuit board 106 that faces away from the UV lamp beads 108. The circuit board 106 is additionally connected to a charging port 162, which is positioned within the plug-in port 158. In this arrangement, the rechargeable battery 160 serves as the primary power source for the multifunctional cosmetic case, supplying electrical energy to all functional modules, including the UV lamp beads 108, the UV camera 110, the display module 112, the touch switch 122, and the speaker 156.

When charging is required, the user connects an external charging cable to the charging port 162 via the plug-in port 158. This design allows convenient recharging of the internal battery without disassembly, thereby maintaining portability and usability. To further enhance user interaction, the speaker 156 may emit audible tones during the charging process. For example, the speaker 156 may provide a first signal indicating charging initiation and a second signal indicating full charge. These audible prompts improve user awareness of the device's power status, ensuring reliability and reducing the likelihood of accidental battery depletion.

In an embodiment, the cosmetic case is provided with an integrated artificial intelligence (AI) analysis module that is operatively coupled to the ultraviolet (UV) camera 110. The AI analysis module is configured to receive, process, and analyze the image data captured by the UV camera 110, enabling advanced skin diagnostics and cosmetic monitoring beyond simple visual feedback. Through image recognition algorithms and predictive modeling, the AI analysis module can detect variations in sunscreen coverage, identify uneven cosmetic application, and assess potential dermatological concerns such as hyperpigmentation or dryness. The results of the analysis are transmitted to the display module in a user-friendly format, such as colored overlays (e.g., blue or red tonal mapping) to enhance visibility across different skin tones, thereby avoiding misinterpretation of the UV imagery.

In addition, the AI analysis module is operatively connected to a speaker 156 integrated within the cosmetic case, which provides audible feedback to the user. The speaker 156 may be configured to emit instructional prompts, alerts, or confirmations, such as notifying the user when sunscreen application is insufficient, when sterilization of an applicator is complete, or when a recommended treatment cycle has ended. By combining visual UV image analysis with real-time audio guidance, the cosmetic case delivers an intuitive and interactive user experience, significantly improving both the accuracy and convenience of cosmetic application and skincare monitoring.

In an embodiment, the multifunctional cosmetic case incorporates a mounting groove 164 on one side of the base box 102. A first mounting portion 166 is provided on one side of the light-transmitting cover plate 114, while a second mounting portion 168 is provided on one side of the cover box 116. The first mounting portion 166 and the second mounting portion 168 are disposed within the mounting groove 164, together defining a mounting cavity. A hinge 170 is positioned within the mounting cavity and is secured to the wall of the mounting groove 164.

The hinge 170 structurally couples the flip cover 104 to the base box 102, enabling relative rotation between the two. Through this configuration, the flip cover 104 can be smoothly opened or closed by the user, thereby facilitating convenient access to both the cosmetic storage area and the integrated electronic modules. Various hinge types may be adopted, including conventional hinges, damping hinges, or spring hinges. In a preferred embodiment, the hinge 170 is embodied as a damping hinge, which provides controlled resistance during opening and closing operations. This design yields a smooth and stable movement, while also enabling the flip cover 104 to automatically maintain an open position at a specific angle. Such functionality improves usability, as the user can operate the cosmetic case hands-free once the flip cover 104 is opened.

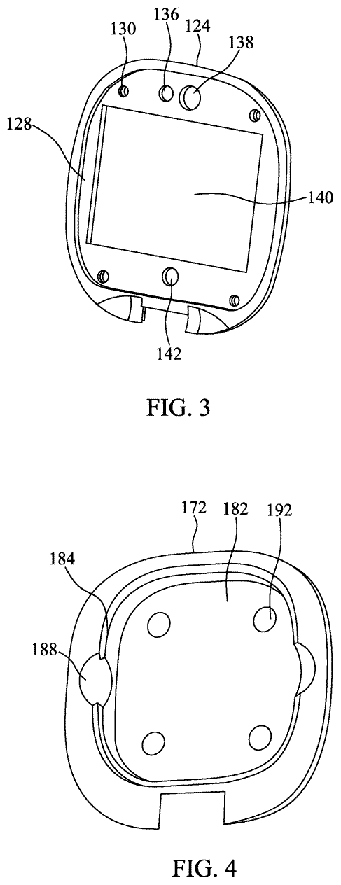

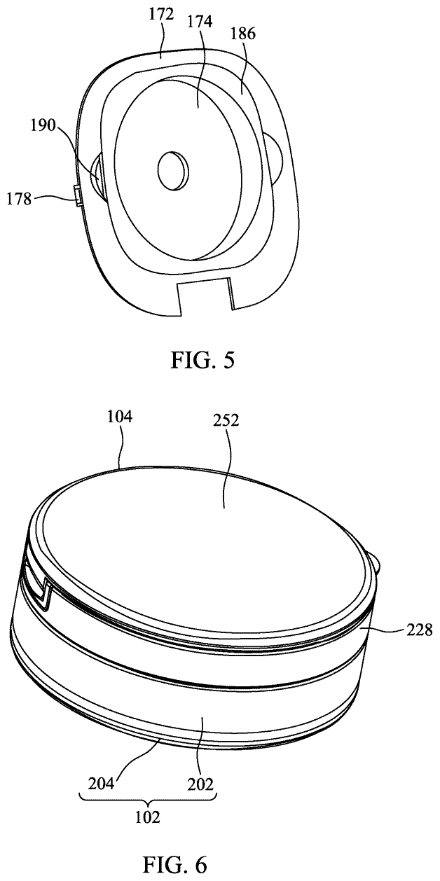

Referring to FIGS. 2, 4, and 5, the base box 102 further comprises an inner shell 172. The inner shell 172 provides an additional internal housing structure and is fabricated from a rigid material such as engineering plastic or metal alloy. The provision of the inner shell 172 significantly increases the overall strength and rigidity of the base box 102, thereby enhancing durability and resistance to mechanical stresses encountered during transportation and daily use.

Inside the inner shell 172, a removable tray 174 is provided. The removable tray 174 is configured to accommodate a powder box 176 or similar cosmetic container. The removable tray 174 is detachable, allowing the user to conveniently remove it from the inner shell 172 for the replacement of cosmetics, cleaning of components, or reconfiguration of storage. The bottom of the removable tray 174 is further provided with a through-hole 180. By pressing through the through-hole 180, the user can eject the powder box 176 from the removable tray 174 with ease, thereby simplifying replacement and maintenance operations.

The edges of the inner shell 172 are provided with a plurality of snap-fitting portions 178, each defining a slot. Corresponding buckles are arranged along the edges of the base box 102. During assembly, the buckles engage the slots of the snap-fitting portions 178, enabling the inner shell 172 to be removably secured to the base box 102. This snap-fit connection provides both mechanical stability and the convenience of disassembly.

In an embodiment, the inner shell 172 includes a placement groove 182. The sidewalls of the placement groove 182 are provided with a step 184. The removable tray 174 is formed with a flange 186 that is received within the placement groove 182, such that the flange 186 rests securely on the step 184. This arrangement ensures that the removable tray 174 remains fixed in position during use, preventing undesired movement or dislodgement.

In addition, hand grip grooves 188 are provided on opposite sides of the inner shell 172. Each hand grip groove 188 extends obliquely outward from the inner side of the step 184 to the top edge of the inner shell 172. The interaction of the flange 186 with the hand grip grooves 188 creates a channel 190 between the flange and the groove. This channel 190 is specifically designed to accommodate the user's fingers, enabling the user to insert their fingers beneath the flange 186 of the removable tray 174. By lifting the flange 186 upward through the channel 190, the user can conveniently remove the removable tray 174 from the placement groove 182. This ergonomic feature eliminates the difficulty of grasping the tray directly, thereby improving user convenience and efficiency during removal and replacement operations.

Furthermore, in an embodiment, the bottom of the placement groove 182 of the inner shell 172 is provided with a plurality of positioning grooves 192, while the bottom of the removable tray 174 is correspondingly provided with positioning protrusions 194. In one embodiment, the positioning grooves 192 are located at the four corners of the placement groove 182. In contrast, the positioning protrusions 194 are arranged at the four corners of the bottom surface of the removable tray 174. When the removable tray 174 is inserted into the inner shell 172, the positioning protrusions 194 engage with the positioning grooves 192, thereby achieving precise alignment. This design simplifies assembly, ensures accurate positioning, and prevents wobbling or misalignment during use.

Collectively, these structural features, including the inner shell 172, the removable tray 174, the flange 186 and step 184 configuration, the hand grip grooves 188 with channel 190, and the positioning grooves 192 with the positioning protrusions 194, provide a robust, ergonomic, and user-friendly system for storing and managing cosmetics within the multifunctional cosmetic case. This design not only improves assembly precision and product stability but also enhances the overall ease of use and hygiene by enabling effortless replacement and cleaning of cosmetic components.

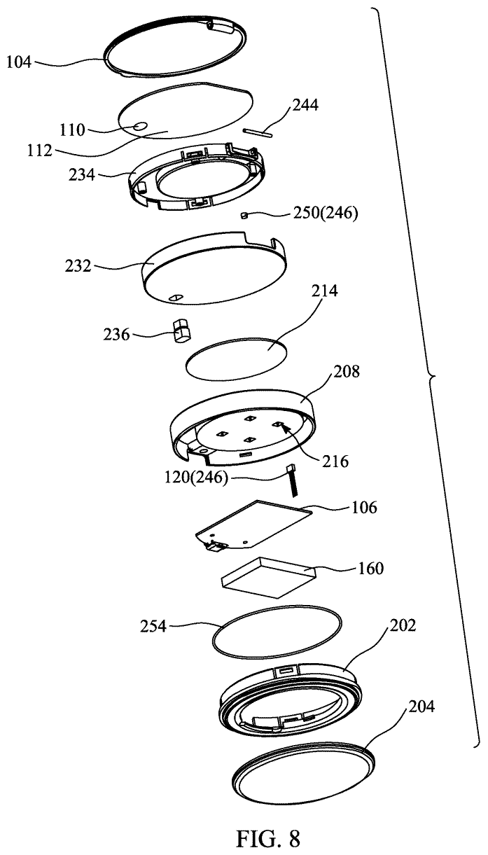

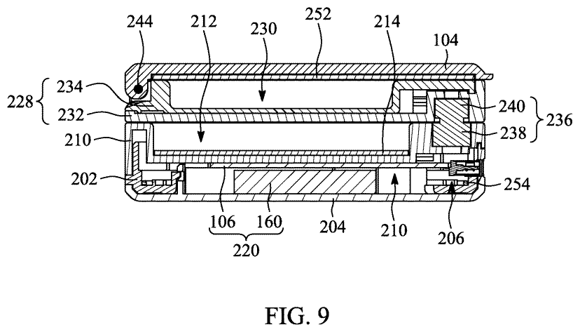

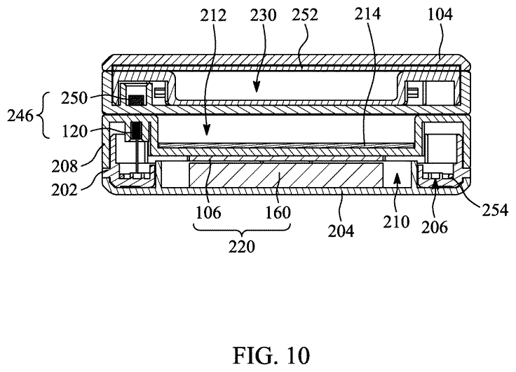

Referring to FIGS. 6 to 10, according to an embodiment of the present invention, another configuration of a multifunctional cosmetic case is provided. A cosmetic case with a powder puff is provided that has an integrated sterilisation and cosmetic storage function. The cosmetic case comprises a base box assembly 200, an electrical assembly 220, a middle box 228, a first rotating shaft 236, a flip cover 104, and a second rotating shaft 244, all cooperatively arranged to provide a compact, portable, and multifunctional cosmetic container.

The base box assembly 200 comprises a base box 102 and a bottom cover 208. The bottom cover 208 is configured to engage with and cover the base box 102 in a detachable or reversible manner, thereby forming an enclosed assembly space 210 for accommodating the electrical assembly 220. In greater detail, the base box 102 comprises a frame 202 and a bottom support plate 204. The frame 202 and the bottom support plate 204 are detachably connected, such as by snap-fit, screw-fit, or other fastening means, to allow assembly and disassembly for maintenance or cleaning. The bottom cover 208 is further provided with a first storage space 212 on the side facing away from the base box 102. The first storage space 212 is specifically dimensioned to accommodate a powder puff and is provided, at least in part, with a light-transmitting window that enables ultraviolet radiation to pass therethrough.

The electrical assembly 220 is securely mounted within the assembly space 210 defined between the base box 102 and the bottom cover 208. The electrical assembly 220 comprises a rechargeable or replaceable battery 160 and a circuit board 106 operatively connected to the rechargeable battery 160 to receive power. The circuit board 106 carries a sterilization module which integrates an ultraviolet (UV) light source 226, which may be formed of one or more UV lamp beads or light-emitting diodes (LEDs). During operation, the sterilization module, having the ultraviolet light source 226, emits ultraviolet light that passes through the light-transmitting window into the first storage space 212, thereby irradiating and sterilizing the powder puff placed therein. In this manner, bacterial and microbial contamination of the powder puff is reduced, thereby enhancing user hygiene and safety.

The middle box 228 is arranged above the base box assembly 200 and is rotatably connected to the base box assembly 200 through the first rotating shaft 236. The first rotating shaft 236 is disposed such that its axis is parallel to the stacking direction of the base box 102 and the bottom cover 208, thereby permitting a smooth rotational motion of the middle box 228 relative to the base box assembly 200. The middle box 228 is provided, on the side facing away from the base box assembly 200, with a second storage space 230. The second storage space 230 is configured to accommodate cosmetic products such as powder compacts, pressed powder, or similar cosmetic substances, thereby enabling the case to simultaneously store both the cosmetic material and the corresponding applicator.

The flip cover 104 is arranged above the middle box 228 and is rotatably coupled to the middle box 228 via the second rotating shaft 244. The second rotating shaft 244 is oriented perpendicular to the axis of the first rotating shaft 236, thereby creating a two-axis rotational mechanism. This allows the flip cover 104 to rotate relative to the middle box 228 to selectively open or close the second storage space 230. When in the closed position, the flip cover 104 effectively seals the second storage space 230, thereby protecting the powder compact stored within from dust, contamination, and unintended spillage.

During use, the cosmetic case provides an integrated dual-storage and sterilization function. A powder puff is placed within the first storage space 212 of the bottom cover 208, and a powder compact is placed within the second storage space 230 of the middle box 228. When the middle box 228 and the bottom cover 208 are in the overlapped and closed configuration, the sterilization module integrated with multiple ultraviolet light sources 226 activates to emit ultraviolet light. The emitted radiation passes through the light-transmitting window and directly illuminates the powder puff, thereby sterilizing and disinfecting the applicator. This ensures that the powder puff remains clean and hygienic during non-use periods.

When the user intends to apply makeup, the middle box 228 is rotated about the first rotating shaft 236 to separate it from the bottom cover 208, thereby exposing the first storage space 212 and permitting removal of the sterilized powder puff. Thereafter, the flip cover 104 is rotated about the second rotating shaft 244 to expose the second storage space 230, thereby providing access to the powder compact. The user may then employ the sterilized powder puff to contact the powder compact and apply makeup onto the skin. This sequence of operations ensures that the applicator contacting the cosmetic material is freshly sterilized, thereby minimizing bacterial contamination and promoting better skin hygiene.

Thus, the cosmetic case of the present embodiment advantageously integrates storage of a powder puff and a powder compact with ultraviolet sterilization, all within a compact and portable structural configuration. By employing the dual-shaft rotation mechanism, the case provides intuitive and user-friendly access to both the applicator and the cosmetic product while simultaneously maintaining the applicator in a hygienic condition. This innovative arrangement addresses the long-standing issue of bacterial contamination in cosmetic applicators and enhances the safety, hygiene, and convenience of daily cosmetic use.

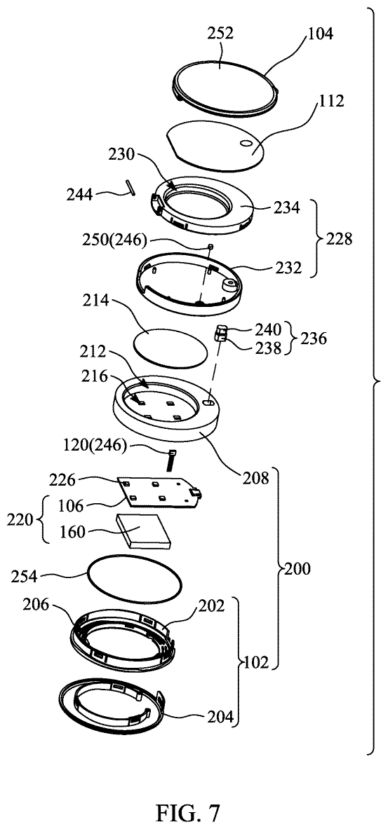

Referring to FIGS. 7 to 10, in an embodiment, the base box assembly 200 of the cosmetic case is further provided with a transparent plate 214 to enhance the sterilization effect and structural integrity of the device. The bottom wall of the first storage space 212 is provided with at least one first light opening 216. Each first light opening 216 is positioned to correspond with at least one ultraviolet light source 226 disposed on the circuit board 106 of the electrical assembly 220. The transparent plate 214 is arranged to cover the first light opening 216, thereby forming a sealed light-transmitting window that allows ultraviolet radiation to pass into the first storage space 212 while simultaneously preventing ingress of dust, powder, or debris.

In an embodiment, when the middle box 228 is rotated into the closed position relative to the first storage space 212, the ultraviolet light source 226 is activated. Ultraviolet radiation emitted by the light source passes through the transparent plate 214 covering the first light opening 216 and enters the first storage space 212. This arrangement allows UV light to be directed upward from the bottom wall of the first storage space 212 toward the powder puff, thereby exposing the puff surface to sterilizing radiation. As a result, microbial contamination is significantly reduced, ensuring that the applicator is maintained in a clean and hygienic state before use.

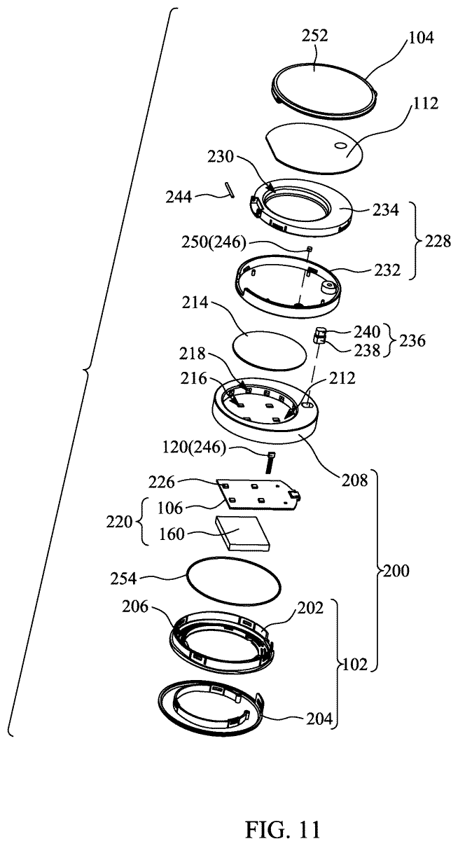

Referring to FIG. 11, in an embodiment, the base box assembly 200 is further provided with a transparent circumferential panel disposed along the inner wall of the first storage space 212. A plurality of second light openings 218 are defined in the circumferential wall of the first storage space 212, spaced apart at predetermined intervals. Each second light opening 218 corresponds to at least one ultraviolet light source 226 mounted on the circuit board 106. The circumferential panel is attached to the inner circumferential wall of the first storage space 212 and covers the second light openings 218 to form a series of circumferential light-transmitting windows. When the middle box 228 closes the first storage space 212, the ultraviolet light source 226 emits radiation that is transmitted through the circumferential panel at each of the second light openings 218, thereby irradiating the powder puff from multiple lateral directions. This circumferential irradiation of the sterilization module improves sterilization coverage by reducing shadowed areas and enabling side-exposure of the puff, which is not fully achieved by bottom irradiation alone.

In an embodiment, both the sterilization module and its configurations are combined for enhanced functionality. Specifically, at least one first light opening 216 is defined in the bottom wall of the first storage space 212, while multiple second light openings 218 are defined around its circumferential inner wall. The base box assembly 200 is correspondingly provided with both the transparent plate 214 to cover the first light opening(s) 216 and a transparent circumferential enclosure to cover the second light openings 218. This dual arrangement allows ultraviolet radiation to be emitted both upward from the bottom wall and radially inward from the circumferential wall, thereby achieving multi-directional exposure. In this configuration, the powder puff is irradiated from below and from the sides simultaneously, ensuring more comprehensive sterilization, effectively eliminating microbial presence even on irregular or recessed surfaces of the puff.

In an embodiment, the electrical assembly 220 includes a control assembly 246 designed to regulate the activation of the ultraviolet light source 226 based on the positional relationship between the middle box 228 and the bottom cover 208. The control assembly 246 is mounted on the base box assembly 200 and/or the middle box 228 and is electrically coupled to the circuit board 106. The control assembly 246 ensures that the UV light source 226 is activated only when the first storage space 212 is fully enclosed and is deactivated immediately when the middle box 228 and the bottom cover 208 are separated. This prevents unintended ultraviolet exposure to the user and conserves power.

Referring to FIGS. 7, 8, and 10, the control assembly 246 comprises a Hall sensor 120 and a magnet 250. The Hall sensor 120 is fixed to the base box 102 and electrically connected to the circuit board 106, while the magnet 250 is mounted on the middle box 228. When the middle box 228 and the bottom cover 208 are in the closed configuration, the Hall sensor 120 and the magnet 250 are positioned in proximity, resulting in the detection of a magnetic field of sufficient strength. This detection triggers the circuit board 106 to energize the ultraviolet light source 226, thereby initiating sterilization. Conversely, when the middle box 228 is rotated away from the bottom cover 208, the relative position of the magnet 250 changes, weakening or removing the magnetic field detected by the Hall sensor 120. As a result, the control assembly 246 deactivates the ultraviolet light source 226, immediately ceasing emission.

The dynamic interaction between the Hall sensor 120 and the magnet 250 provides a highly reliable and contactless control mechanism, as changes in magnetic field strength are precisely detected and used to regulate the operation of the UV source. This ensures that the sterilization process is automatically and seamlessly initiated or terminated, depending on the closed or open state of the cosmetic case.

In an embodiment, the control assembly 246 may be implemented using other types of switches or detection mechanisms. For example, instead of a Hall sensor and magnet, a mechanical contact switch may be used. In such a case, the switch is positioned at an interface between the middle box 228 and the bottom cover 208. When the two components are in the closed position, the contact switch is engaged, thereby completing the circuit and activating the ultraviolet light source 226. When the middle box 228 and the bottom cover 208 are separated, the contact switch is disengaged, breaking the circuit and deactivating the ultraviolet light source 226. Such embodiments, whether magnetic or mechanical, ensure user safety by preventing ultraviolet emission in an open state and by enabling sterilization only within an enclosed chamber.

According to an embodiment of the present invention, as illustrated in FIGS. 6 to 10, the middle box 228 may be constructed as a modular assembly including a first box body 232 and a second box body 234. The first box body 232 and the second box body 234 are detachably connected, thereby enabling convenient assembly, disassembly, and reconfiguration according to the user's needs. In this arrangement, the first box body 232 is rotatably connected to the bottom cover 208 by way of a first rotating shaft 236, while the flip cover 104 is pivotally connected to the second box body 234 via a second rotating shaft 244. The second box body 234 defines a second storage space 230 on the side facing away from the first box body 232, the storage space being dimensioned to accommodate a powder compact or similar cosmetic product.

This detachable arrangement permits the cosmetic case to be divided into two independent functional units. One functional unit is configured to hold and sterilize the powder puff and comprises the first box body 232, the base box assembly 200, the first rotating shaft 236, and the electrical assembly 220. Another functional unit is configured to hold and protect the powder compact and comprises the second box body 234, the flip cover 104, and the second rotating shaft 244. Such a configuration offers the user enhanced flexibility and portability, as the user may selectively carry only the puff-holding part or only the compact-holding part, depending on personal requirements. For instance, when hygiene maintenance of the puff is of primary importance, the user may carry only the first functional unit. Alternatively, when only cosmetic replenishment is required, the user may carry solely the second functional unit.

In an embodiment, the middle box 228 may be integrally molded as a single component. The integrally molded middle box 228 can be manufactured using an injection molding process, which ensures high production efficiency, improved structural consistency, and precise dimensional tolerances.

Similarly, in an embodiment, the frame 202, the bottom support plate 204, and the flip cover 104 of the base box assembly 200 may also be integrally formed by injection molding. This integral molding process reduces the number of assembly steps, lowers manufacturing cost, and provides superior overall rigidity and durability to the cosmetic case.

Referring to FIGS. 7 to 9, in an embodiment, the first rotating shaft 236 comprises a first shaft segment 238 and a second shaft segment 240. The first shaft segment 238 and the second shaft segment 240 are coupled in a damped rotational configuration, thereby allowing controlled relative motion between the middle box 228 and the bottom cover 208. Specifically, the first shaft segment 238 is mounted to the first box body 232, and the second shaft segment 240 is mounted to the bottom cover 208. Due to the damping effect produced by the interface between the shaft segments, the user experiences a tactile resistance when rotating the middle box 228 relative to the base box assembly 200. This damping effect not only provides a premium operational feel but also prevents the middle box 228 from rotating too freely, thereby reducing the risk of accidental opening during handling or transport. Furthermore, the damping action stabilizes the powder puff stored in the first storage space 212, preventing it from being dislodged and ensuring hygienic protection at all times.

In yet another embodiment, as depicted in FIGS. 7 to 10, the cosmetic case is provided with a makeup mirror 252 mounted on the second side of the flip cover 104. This integrated makeup mirror 252 allows the user to conveniently perform cosmetic application without the need for a separate mirror. The strategic placement of the mirror within the flip cover 104 ensures that the user can readily access both the powder compact in the second storage space 230 and their own reflection, thereby streamlining the makeup process. The inclusion of this mirror enhances the functional value of the cosmetic case, making it an all-in-one cosmetic tool.

The cosmetic case is further configured with a dual-functional flip cover 104 that provides both imaging and mirror-based functionality. The first side (inner side) of the flip cover is provided with an integrated display module 112, a UV light source 226 integrated with a plurality of UV lamp beads 108, and a UV camera 110. The UV light source 226 is oriented to emit ultraviolet radiation toward the user's skin, while the UV camera 110 is positioned to capture reflected UV light from the illuminated surface. The captured image data is processed and displayed in real time on the display module, allowing the user to visually assess sunscreen application, cosmetic coverage, or underlying skin conditions that are otherwise invisible under natural lighting. The integration of these elements within the flip cover ensures compactness, portability, and ease of access while maintaining the high performance of a dedicated imaging device.

The second side (outer side) of the flip cover is provided with a makeup mirror 252, which is encircled by a shooting light ring configured to operate in at least two modes: a visible illumination mode and an infrared (IR) therapy mode. In the visible illumination mode, the light ring emits uniform white light to enhance brightness and clarity during cosmetic application, photography, or video recording. In the IR therapy mode, the light ring emits non-visible infrared radiation within a wavelength range of approximately 850-940 nm, which has been demonstrated to be safe for ocular exposure and effective for therapeutic purposes such as alleviating dry eye symptoms, promoting skin rejuvenation, and reducing inflammation. The strategic placement of the mirror with the surrounding light ring provides both cosmetic utility and therapeutic benefits, thereby extending the functionality of the cosmetic case beyond conventional storage and application.

In an embodiment, the cosmetic case is further enhanced with an artificial intelligence (AI) module that cooperates with the UV camera 110 and display module 112. The AI analysis module is configured to process the UV images, identify variations in cosmetic distribution, detect regions of insufficient sunscreen application, and provide predictive insights regarding potential skin conditions such as hyperpigmentation, uneven tone, or premature aging. The AI analysis module may further generate color-coded overlays on the display, such as red or blue highlights, to intuitively represent areas requiring attention. This not only overcomes limitations of traditional grayscale UV imaging but also ensures inclusivity across diverse skin tones by providing clear and interpretable feedback to all users.

The integration of the dual-sided flip cover 104 structure with UV imaging, therapeutic illumination, and AI-driven skin analysis enables the cosmetic case to serve as a multifunctional skincare companion. A user may employ the first side of the flip cover 104 for real-time monitoring of sunscreen or makeup effectiveness, while the second side of the flip cover 104 can be used for conventional cosmetic application under enhanced illumination or for receiving therapeutic IR exposure. The AI-supported diagnostic capability further elevates the cosmetic case into a smart device, offering personalized recommendations, reminders for sunscreen reapplication, and predictive skincare monitoring. This embodiment thus combines storage, imaging, therapy, and intelligent feedback into a single compact housing, addressing both aesthetic and dermatological needs in a portable and user-friendly format.

The flip cover is configured to be rotatable or flippable, thereby enabling the user to selectively alternate between the display side and the mirror side according to their specific needs.

Moreover, the cosmetic case may be equipped with a decorative light strip 254 to improve both aesthetics and user appeal. The frame 202 of the base box 102 is provided with circumferential mounting grooves 206, and the bottom support plate 204 is configured with a light-transmitting area corresponding to the mounting grooves 206. The decorative light strip 254 is installed within the circumferential mounting grooves 206 and electrically connected to the circuit board 106. The decorative light strip 254 may comprise various types of lighting, including but not limited to colored illumination, neon lighting, or dynamic breathing lights, thereby offering multiple decorative effects.

When activated, the decorative light strip 254 emits light that passes through the light-transmitting region of the bottom support plate 204, producing a visually striking effect that decorates the cosmetic case. This design not only enhances the external appearance and attractiveness of the cosmetic case but also serves as a functional indicator of device operation, charging status, or sterilization progress, depending on implementation. The addition of the decorative light strip thus contributes to both the aesthetic enhancement and practical user experience of the cosmetic case.

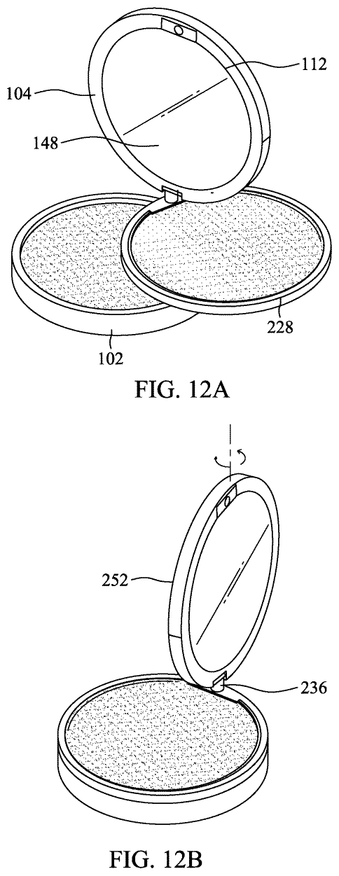

According to an embodiment of the present invention, referring to FIGS. 12A and 12B, an alternate configuration of a cosmetic case is provided which incorporates a unique flip cover assembly configured not only to open and close conventionally, but also to rotate about its own central axis when in the open position. This structural innovation allows the inner surface of the flip cover, which is equipped with a display and UV imaging components, to be selectively oriented for either internal or external use, thereby greatly enhancing the multifunctionality of the cosmetic case.

The cosmetic case comprises a base box 102 defining a middle box 228 configured to accommodate a cosmetic product, such as powder, compact foundation, or other makeup material. A flip cover 104 is connected to the base box 102 via a hinge or a first rotating shaft 236, allowing the flip cover to pivot between a closed position, in which the middle box 228 is sealed, and an open position, in which the middle box 228 is exposed for user access.

Unlike conventional cosmetic cases, the flip cover 104 is further configured with an additional rotational capability about its own central axis when in the open position. In practice, when the flip cover 104 is flipped open to expose the middle box 228, the flip cover can be rotated circumferentially relative to the base box 102. This rotation enables the display area 148 integrated on the inner surface of the flip cover 104 to be selectively reoriented such that, upon reclosing the flip cover, the display area 148 faces outward instead of inward.

The display area 148 disposed on the flip cover 104 is operatively connected to an electrical assembly embedded within the cosmetic case. In an embodiment, the display area 148 serves as a user interface for UV imaging functions. Specifically, the flip cover 104 is equipped with a UV camera 110 and one or more UV lamp beads 108 strategically positioned adjacent to the display. When the flip cover is rotated and subsequently closed in the outward-facing orientation, the UV camera 110 and UV lamp beads 108 become externally exposed on the outer surface of the case. This unique structural configuration allows the user to perform UV imaging of their facial skin without the need to open the cosmetic case or expose its contents.

During operation, the user initiates UV imaging by activating the control assembly 246 of the cosmetic case. The UV light sources emit ultraviolet radiation toward the user's skin surface, while the UV camera captures reflected UV images. These images are transmitted to the display area 148 integrated on the flip cover 104. As the flip cover has been rotated to face outward, the display is directly visible to the user even when the cosmetic case is closed, thereby allowing real-time feedback and analysis of skin conditions such as sunscreen coverage, cosmetic distribution, or detection of blemishes and pigmentation not visible under ordinary light.

This arrangement provides several technical advantages. First, the user can conveniently access UV imaging and skin analysis functions without repeatedly opening and closing the cosmetic case, thus preventing contamination of stored makeup products. Second, the ability to reorient the flip cover ensures that the multifunctional imaging module can serve dual purposes: (i) when the cover is oriented inward, it functions as an internal display for aiding cosmetic application with the case open; and (ii) when rotated outward, it operates as an external diagnostic tool for UV imaging with the case closed. Third, by maintaining the compact and portable structure of a conventional cosmetic case, the invention provides multifunctional benefits without significantly increasing bulk or complexity.

In an embodiment, the rotational connection between the flip cover 104, middle box 228, and the base box 102 may incorporate a detent mechanism, torque-limiting structure, or magnetic alignment system to ensure smooth rotation and stable positioning of the flip cover in the outward-facing orientation. Furthermore, locking structures may be provided to secure the flip cover in the desired rotational alignment before closure, thereby ensuring that the display, UV light sources, and UV camera remain optimally oriented for external operation.

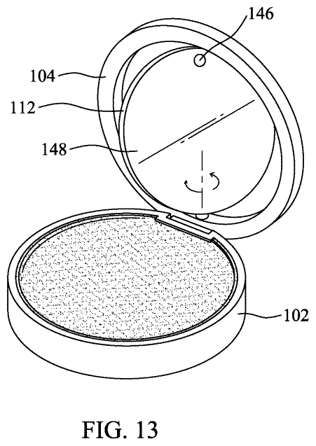

Referring to FIG. 13, in an embodiment, an alternate configuration of the multifunctional cosmetic case is provided. The cosmetic case has a unique flip cover 104 that integrates both a display area 148 and a makeup mirror 252, with the ability to interchange their positions through rotational movement. This dual-surface configuration allows the user to selectively expose either the display area 148 or the makeup mirror 252 to the inside or the outside of the cosmetic case, depending on the functional requirement at any given time.

The cosmetic case comprises a base box 102 defining a storage cavity for accommodating a cosmetic product, such as a pressed powder, sunscreens, foundation, etc., or makeup tools such as a powder puff. The flip cover 104 is pivotally connected to the base box 102 via a hinge structure, thereby enabling the flip cover to move between an open position, in which the storage cavity is accessible, and a closed position, in which the cavity is sealed. Unlike conventional cosmetic cases, the flip cover 104 of the present invention is equipped with a rotationally mounted frame that allows internal components to be repositioned.

In an embodiment, the flip cover 104 may be an annular frame structure within which a rotatable sub-frame is housed. The sub-frame supports a UV camera 110 and a display module 112 that has a display area 148 on one surface and a makeup mirror 252 on the opposite surface. The display module 112 is mounted such that it can rotate about its central axis relative to the flip cover 104. Through this design, the display area 148 and the makeup mirror 252 can be interchanged between an inward-facing orientation (toward the cosmetic cavity) and an outward-facing orientation (toward the external environment).

The display area 148 is electrically connected to a circuit board located within the base box 102 or within the flip cover 104 itself, and is equipped with ultraviolet (UV) imaging components, including a UV camera 110 and one or more UV lamp beads 108. These components are arranged in proximity to the display area 148 and are configured to irradiate the user's skin with UV light and capture reflected images when the display is oriented outward. The captured images are then processed and displayed on the screen, allowing the user to analyze sunscreen application, cosmetic distribution, or skin conditions invisible under natural light.

On the opposite side of the display area 148, the makeup mirror 252 serves as a traditional cosmetic mirror. When the display area 148 is rotated such that the makeup mirror 252 faces inward, the user can utilize it for conventional cosmetic application while accessing the stored cosmetic product within the base box 102. Conversely, when rotated outward, the makeup mirror 252 can be used independently of the cosmetic case contents, for example, during quick adjustments or grooming.

This interchangeable configuration provides the user with flexibility to select which surface (mirror or display) faces inward or outward, depending on the mode of use. For example, when the user desires to apply makeup, the makeup mirror 252 may be rotated inward to reflect while the case is open. When the user wishes to analyze their skin using UV imaging, the display area 148 may be rotated outward, thereby exposing the imaging components for external use while keeping the cosmetic compartment sealed and protected.

In an embodiment, detent mechanisms, magnetic or hall sensor locking arrangements, or frictional interfaces are provided between the flip cover 104 and the display area 148 to secure the display module 112 or makeup mirror 252 in a stable position after rotation. Such mechanisms prevent unintentional rotation during handling, ensuring precise alignment of the chosen surface. Additionally, electrical contacts or flexible connectors are provided to maintain a stable power supply and data transmission to the display module 112 regardless of its rotational orientation.

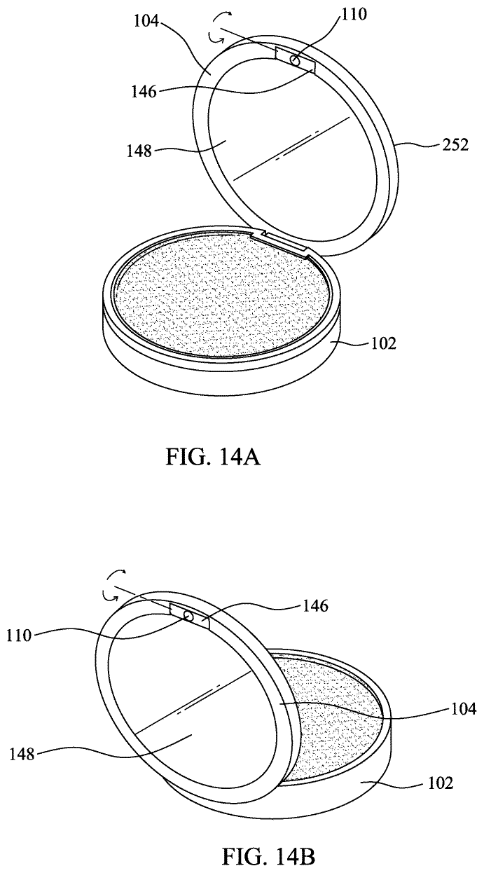

Referring to FIGS. 14A and 14B, in an embodiment of the present invention, yet another configuration of a multifunctional cosmetic case is disclosed that incorporates a fixed display module 112 with a display area 148 in cooperation with a rotatable camera area 146 mounted along the circumference of the flip cover 104. The cosmetic case comprises a base box 102 configured for storing one or more cosmetic products, and a flip cover 104 hingedly connected to the base box 102. The flip cover 104 houses the display area 148, which may be positioned on the inner side, the outer side, or on both sides of the flip cover, depending upon the configuration of the embodiment. Specifically, the display area 148 remains fixed to the flip cover 104, thereby ensuring consistent operability and durability during repeated use.

The camera area 146 has a UV camera 110 provided on the circumferential edge of the flip cover 104 and is coupled via a rotational support mechanism to the flip cover frame. This configuration permits the camera area 146 to rotate independently of the display area 148, such that the UV camera 110 may be directed toward the inner side of the flip cover 104 or toward the outer side of the flip cover 104. In an inner-facing orientation, when the flip cover 104 is opened relative to the base box 102, the camera area 146 is oriented inward to face the user. In this mode, the user may apply cosmetic products stored within the base box 102 while simultaneously observing their face on the display area 148, which is operatively connected to the camera area 146. The integration of ultraviolet (UV) lamp beads adjacent to the UV camera 110 further allows the user to obtain high-contrast imaging of cosmetic coverage, sunscreen uniformity, or residual impurities on the skin surface during cosmetic application.

In contrast, when the user desires to conduct skin analysis or sunscreen detection without accessing the cosmetic storage compartment, the camera area 146 may be rotated to the outer-facing orientation. In this configuration, the flip cover 104 is closed against the base box 102, and the outwardly oriented UV camera 110 captures the user's facial features without the need to open the cosmetic case. The display area 148, depending on its placement, may then render the captured image in either visible or ultraviolet imaging modes, thereby allowing the user to evaluate sunscreen effectiveness, monitor cosmetic residue, or inspect skin conditions. This dual-orientation capability of the camera area 146, while maintaining the display area 148 in a fixed configuration, offers an unprecedented combination of convenience, compactness, and multifunctionality within a single cosmetic case.

In an embodiment, the rotational mounting of the camera area 146 may be configured with a step-lock, magnetic lock mechanism, a damping hinge assembly, or a rotary encoder system. Such configurations not only ensure controlled and stable repositioning of the UV camera 110 but may also be integrated with an electronic positional sensing unit, such as a Hall-effect sensor or a microswitch, operatively linked to the case's control circuit. In operation, when the control circuit detects that the camera area 146 has been rotated inward, the system may default to a cosmetic application mode, presenting magnified visible light imagery on the display. Conversely, when the camera area 146 is rotated outward, the system may automatically activate a diagnostic mode, switching the display to render UV or colour-inverted images that highlight sunscreen residues or underlying skin features. By separating the rotational mobility of the camera area 146 from the fixed configuration of the display area 148, this arrangement achieves a robust, user-friendly, and adaptable cosmetic case that integrates storage, imaging, and diagnostic functionalities into one compact system.

In an embodiment, the multifunctional cosmetic case may be integrated with an LED light ring, configured as a dual-mode lighting system, wherein the same annular array of LEDs may be selectively switched between invisible IR therapy mode and visible white light illumination mode. In the visible mode, the light ring emits uniform, high-intensity white light that evenly illuminates the user's face, thereby facilitating precise makeup application, professional photography, or video recording. When switched to therapeutic mode, the white light is deactivated and the IR diodes are activated, delivering non-visible irradiation for skin therapy or eye comfort without interfering with the user's visual field. This dual-function system thus enables the user to simultaneously apply makeup under optimal illumination while discreetly receiving therapeutic exposure, ensuring convenience, efficiency, and multifunctionality within a single device.

The dual-mode operation of the LED light ring may be governed by a control assembly electrically connected to the cosmetic case's circuit board. The control assembly can include touch-sensitive activation zones or sensor-based switching mechanisms mounted on the flip cover, allowing the user to seamlessly toggle between visible illumination and therapeutic IR emission. Safety measures may be further incorporated, such as automatic deactivation of IR emission when the mirror is positioned too close to the user's eyes, achieved through the use of a proximity sensor or distance detection module. Such integration ensures that the therapeutic function is both effective and inherently safe, maintaining compliance with applicable optical radiation exposure standards.

In an embodiment of the present invention, the multifunctional cosmetic case is configured with a colour inversion imaging system that enables the display module to render ultraviolet (UV) reflection data in selectable colour palettes, rather than the conventional black-and-white output. Traditional monochrome UV imaging systems rely on grayscale rendering, which often produces ambiguous results, especially for users with darker skin tones, where naturally pigmented areas appear uniformly dark. This can lead to misinterpretation of cosmetic coverage or hygiene, as healthy regions of skin may appear indistinguishable from areas requiring cosmetic application. The present invention addresses this deficiency by introducing colour inversion processing, wherein UV reflectance intensities are mapped to vivid, high-contrast colour schemes such as red-blue gradients, heat maps, or false-colour UV overlays.

In an embodiment, the display module 112 may be configured to operate in multiple modes. For example, in a red-blue inversion mode, higher reflectance areas are displayed in bright blue, while lower reflectance areas are displayed in red, thereby enhancing contrast across all skin types. In another mode, a heat-map gradient ranging from green to yellow to red is used to indicate varying cosmetic or sunscreen thickness levels. A false-colour UV mode may also be provided, mapping UV reflectance into unique colour scales such as violet and cyan. These colour inversion modes are selectable by the user through an interface on the display, allowing intuitive customization based on preference and situational needs.

In an embodiment of the present invention, the multifunctional cosmetic case is configured not merely as a storage device but as an integrated diagnostic, therapeutic, and cosmetic application platform. The cosmetic case incorporates a UV imaging module cooperatively interfaced with an ultraviolet (UV) camera and artificial intelligence (AI) algorithms. This arrangement enables real-time analysis of the user's skin under ultraviolet irradiation. Importantly, the AI is configured to perform predictive modeling of dermatological conditions, such as forecasting the onset or progression of hyperpigmentation, sun-induced damage, or cosmetic residue buildup. By processing UV reflectance data, the AI generates outputs in colour-inverted imaging schemes (e.g., red-blue) that provide heightened clarity compared to traditional monochrome displays. This allows users of all skin tones to obtain equitable diagnostic insights, thereby addressing inclusivity challenges inherent in conventional UV analysis.

Additionally, the multifunctional cosmetic case integrates a UV index and SPF evaluation system. A dedicated UV sensor is embedded within the housing and configured to continuously detect the ambient UV index in the user's surrounding environment. Based on these readings, the system calculates the recommended duration for sunscreen effectiveness and can alert the user when reapplication of sunscreen is advisable. In an embodiment, the AI cooperates with the UV camera to perform a direct SPF measurement of sunscreen applied on the user's skin, quantifying its protective efficacy over time. This enables a unique feedback loop where the device not only evaluates sunscreen application visually but also tracks its degradation dynamically, ensuring users receive accurate and actionable guidance for maintaining skin protection throughout the day.

In an embodiment, in order to enhance user comfort and hygiene, the cosmetic case may further be equipped with cooling and cleaning mechanisms. The cooling element, such as a thermoelectric Peltier module or a micro-fan assembly, may be embedded adjacent to the mirror or display unit. This cooling module can be selectively activated during UV imaging or phototherapy sessions, thereby refreshing the user's skin and minimizing discomfort associated with prolonged light exposure. Moreover, an integrated antibacterial cleaning feature may be provided within the storage of the cosmetic case. This feature utilizes a UV sterilization process with the help of a UV light source, ensuring that powder puffs, sponges, or brushes remain free of microbial contamination. Together, these features extend the functionality of the cosmetic case beyond diagnostic analysis into the domains of skin wellness and applicator hygiene, establishing a holistic cosmetic management system.

In an embodiment, the multifunctional cosmetic case further incorporates a removable camera module and an integrated applicator system, thereby enhancing portability and user convenience. In some embodiments, the UV camera and its associated imaging optics are mounted on a detachable module that can be separated from the cosmetic case and used independently, for instance, as a handheld skin analysis tool. This allows the user to perform imaging on hard-to-reach body areas while still relying on the main housing for data processing and display. Complementarily, a built-in applicator mechanism is provided for sunscreen or cosmetic products. This applicator may be selectively extended or retracted and, in certain embodiments, may be automatically dispensed with precise dosage control based on the AI's calculation of the user's SPF needs. By integrating diagnostic imaging, predictive modeling, environmental sensing, cooling, cleaning, modular cameras, and application mechanisms into a single platform, the present invention establishes a new paradigm in multifunctional cosmetic systems, surpassing conventional storage cases and standalone imaging devices.

By enabling images to be displayed in vivid, adjustable colours instead of grayscale, this embodiment not only enhances diagnostic clarity but also improves user acceptance and commercial viability. Cosmetic inconsistencies, sunscreen coverage gaps, or impurities are displayed with clear differentiation, independent of natural skin colour. The ability to rotate the flip cover and expose the display externally further allows users to perform UV imaging and review results conveniently without opening the cosmetic case. Collectively, these features transform the cosmetic case into a comprehensive, inclusive, and market-ready diagnostic tool that overcomes the limitations of prior art and provides a superior user experience.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A multifunctional cosmetic case, comprising:
a base box configured to store one or more cosmetic products;
a flip cover coupled to the base box;
an ultraviolet (UV) light source positioned on the flip cover to direct UV light toward a user's skin;
a UV camera configured to capture one or more images of the user's skin irradiated by the UV light; and
a display module operatively connected to the UV camera and arranged to display the captured UV image; and
wherein the multifunctional cosmetic case enables monitoring of application or removal of cosmetic products on the skin.

2. The multifunctional cosmetic case of claim 1, further comprising:
an artificial intelligence analysis module configured to process the one or more images captured by the UV camera to evaluate cosmetic coverage, skin condition, or sun protection factor effectiveness; and
a feedback interface on the display module configured to provide visual or audio feedback to a user based on the artificial intelligence analysis module.

3. The multifunctional cosmetic case of claim 1, further comprising a sensor configured to automatically deactivate the UV light source and the UV camera when the flip cover is closed.

4. A multifunctional cosmetic case, comprising:
a base box having a storage chamber for a cosmetic applicator;
an ultraviolet (UV) sterilization module comprising one or more UV light sources arranged to emit UV light into the storage chamber through a light-transmitting window provided on the storage chamber;
a flip cover having a first side and a second side, a UV camera and a display module provided on the first side, the UV camera is configured to capture one or more images of a user's skin irradiated by the UV light; and the display module is operatively connected to the UV camera to display the captured one or more images; and
a control circuit configured to activate the UV sterilization module when an applicator is enclosed in the storage chamber.

5. The multifunctional cosmetic case of claim 4, further comprising a cosmetic mirror provided on a second side of the flip cover.

6. The multifunctional cosmetic case of claim 4, wherein the UV sterilization module is configured to emit UV light from both a bottom wall and a circumferential wall of the storage chamber.

7. The multifunctional cosmetic case of claim 4, further comprising:
an artificial intelligence analysis module configured to process the one or more images captured by the UV camera to evaluate cosmetic coverage, skin condition, or sun protection factor effectiveness; and
a feedback interface on the display module configured to provide visual or audio feedback to a user based on the artificial intelligence analysis module.

8. The multifunctional cosmetic case of claim 7, further comprising a speaker electrically connected to the control circuit for providing audio feedback.

9. The multifunctional cosmetic case of claim 7, wherein the artificial intelligence analysis module is configured to perform colour inversion imaging to enhance visibility of cosmetic residues or uneven application.

10. The multifunctional cosmetic case of claim 7, wherein the artificial intelligence analysis module evaluates UV index exposure and recommends appropriate SPF usage.

11. The multifunctional cosmetic case of claim 7, wherein the artificial intelligence analysis module includes predictive modeling configured to track changes in a user's skin condition over time.

12. The multifunctional cosmetic case of claim 4, wherein the flip cover is mounted on one or more shafts, allowing two-axis rotation between the first side and the second side.

13. The multifunctional cosmetic case of claim 4, wherein the flip cover comprises a rotatable area with the first side and the second side, and the flip cover is configured to interchange position of the first side and the second side through rotational movement.

14. The multifunctional cosmetic case of claim 4, wherein the flip cover further comprising a rotatable camera area allowing adjustment of the UV camera orientation relative to a user.

15. The multifunctional cosmetic case of claim 4, wherein the UV camera is detachable from the multifunctional cosmetic case and operatively connectable via a wired or wireless interface.

16. A method of cosmetic application and skin analysis using a multifunctional cosmetic case, comprising:
storing at least one cosmetic product in the multifunctional cosmetic case;
irradiating ultraviolet (UV) light toward a user's face from a UV light source provided on the multifunctional cosmetic case;
capturing one or more images of the face irradiated with the UV light using a UV camera;
processing the captured one or more images with an Artificial intelligence analysis module to determine cosmetic distribution, skin features, or SPF effectiveness; and

17. The method of claim 16, wherein capturing the one or more images further comprises detecting variations in cosmetic distribution on a user's skin.

18. The method of claim 16, further comprising measuring the ambient UV index and recommending an SPF value to the user via the display module.

19. The method of claim 16 further comprising: performing colour inversion imaging to enhance visibility of cosmetic residue or uneven application.

20. The method of claim 16, wherein the artificial intelligence analysis module includes predictive modelling configured to track changes in a user's skin condition over time.
